(19) United States
(12) Patent Application Publication
(10) Pub. No.: US 2004/0038214 A1
(43) Pub. Date: Feb. 26, 2004

(54) Method and system for reading a molecular array

(76) Inventor: John F. Corson, Mountain View, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/226,670

(22) Filed: Aug. 23, 2002

(51) Int. Cl. C12Q 1/68; G01N 33/53

(52) U.S. Cl. 435/6; 435/7.1

(57) ABSTRACT

A method and system for accurately scanning the features of a molecular array in order to take into account chromophore chemical decomposition, peak broadening, and other spectral changes that can alter the relationship between the density, or concentration, of chromophore label initially present within a feature of a molecular array and the intensity of light emitted from the feature of the molecular array at a wavelength characteristic of fluorescent emission from the chromophore label. In one embodiment, a four-channel molecular array scanner is used to more accurately scan a two-channel molecular array. All four detectors of a four-channel molecular array scanner are employed to measure signal from a two-channel array. The additional signals may be used to directly measure chromophore decomposition products on the surface of the molecule array, peak broadening, low signal-to-noise-ration signals, or other phenomena that alter the relationship between the initial density, or concentration, of a chromophore label and the fluorescent emission later detected by a molecular array scanner. In alternative embodiments, a greater number of signals are measured and used than the number of types of labels to which a molecular array is exposed in order to more accurately determine initial concentrations of labels in features of the molecular array.

### TECHNICAL FIELD

The present invention is related to molecular array scanners and, in particular, to a method and system for scanning a molecular array to evaluate the binding of labeled target molecules to features of the molecular array by detecting and processing a greater number of signals than the number of types of target-molecule labels applied to the array.

### BACKGROUND OF THE INVENTION

The present invention is related to acquisition of molecular-array data and other types of genetic, biochemical, and chemical data from molecular arrays by molecular array scanners. A general background of molecular-array technology is first provided, in this section, to facilitate discussion of the scanning techniques described in following sections. Molecular arrays are also referred to as “microarrays” and simply as “arrays” in the literature. Molecular arrays are not arbitrary regular patterns of molecules, such as occur on the faces of crystalline materials, but, as the following discussion shows, are manufactured articles specifically designed for analysis of solutions of compounds of chemical, biochemical, biomedical, and other interests.

Array technologies have gained prominence in biological research and are likely to become important and widely used diagnostic tools in the healthcare industry. Currently, molecular-array techniques are most often used to determine the concentrations of particular nucleic-acid polymers in complex sample solutions. Molecular-array-based analytical techniques are not, however, restricted to analysis of nucleic acid solutions, but may be employed to analyze complex solutions of any type of molecule that can be optically or radiometrically scanned or read and that can bind with high specificity to complementary molecules synthesized within, or bound to, discrete features on the surface of an array. Because arrays are widely used for analysis of nucleic acid samples, the following background information on arrays is introduced in the context of analysis of nucleic acid solutions following a brief background of nucleic acid chemistry.

Deoxyribonucleic acid (“DNA”) and ribonucleic acid (“RNA”) are linear polymers, each synthesized from four different types of subunit molecules. The subunit molecules for DNA include: (1) deoxy-adenosine, abbreviated “A,” a purine nucleoside; (2) deoxy-thymidine, abbreviated “T,” a pyrimidine nucleoside; (3) deoxy-cytosine, abbreviated “C,” a pyrimidine nucleoside; and (4) deoxy-guanosine, abbreviated “G,” a purine nucleoside. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1 can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand.

FIGS. 2A-B illustrates the hydrogen bonding between the purine and pyrimidine bases of two anti-parallel DNA strands. AT and GC base pairs, illustrated in FIGS. 2A-B, are known as Watson-Crick (“WC”) base pairs. Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to reannealing of the DNA duplex.

The ability to denature and renature double-stranded DNA has led to the development of many extremely powerful and discriminating assay technologies for identifying the presence of DNA and RNA polymers having particular base sequences or containing particular base subsequences within complex mixtures of different nucleic acid polymers, other biopolymers, and inorganic and organic chemical compounds. One such methodology is the array-based hybridization assay. FIGS. 4-7 illustrate the principle of the array-based hybridization assay. An array (402 in FIG. 4) comprises a substrate upon which a regular pattern of features is prepared by various manufacturing processes. The array 402 in FIG. 4, and in subsequent FIGS. 5-7, has a grid-like 2-dimensional pattern of square features, such as feature 404 shown in the upper left-hand corner of the array. Each feature of the array contains a large number of identical oligonucleotides covalently bound to the surface of the feature. These bound oligonucleotides are known as probes. In general, chemically distinct probes are bound to the different features of an array, so that each feature corresponds to a particular nucleotide sequence. In FIGS. 4-6, the principle of array-based hybridization assays is illustrated with respect to the single feature 404 to which a number of identical probes 405-409 are bound. In practice, each feature of the array contains a high density of such probes but, for the sake of clarity, only a subset of these are shown in FIGS. 4-6.

Once an array has been prepared, the array may be exposed to a sample solution of target DNA or RNA molecules (410-413 in FIG. 4) labeled with fluorophores, chemiluminescent compounds, or radioactive atoms 415-418. Labeled target DNA or RNA hybridizes through base pairing interactions to the complementary probe DNA, synthesized on the surface of the array. FIG. 5 shows a number of such target molecules 502-504 hybridized to complementary probes 505-507, which are in turn bound to the surface of the array 402. Targets, such as labeled DNA molecules 508 and 509, that do not contains nucleotide sequences complementary to any of the probes bound to array surface do not hybridize to generate stable duplexes and, as a result, tend to remain in solution. The sample solution is then rinsed from the surface of the array, washing away any unbound-labeled DNA molecules. In other embodiments, unlabeled target sample is allowed to hybridize with the array first. Typically, such a target sample has been modified with a chemical moiety that will react with a second chemical moiety in subsequent steps. Then, either before or after a wash step, a solution containing the second chemical moiety bound to a label is reacted with the target on the array. After washing, the array is ready for data acquisition by scanning or reading. Biotin and avidin represent an example of a pair of chemical moieties that can be utilized for such steps.

Finally, as shown in FIG. 6, the bound labeled DNA molecules are detected via optical or radiometric scanning or reading. Optical scanning and reading both involve exciting labels of bound labeled DNA molecules with electromagnetic radiation of appropriate frequency and detecting fluorescent emissions from the labels, or detecting light emitted from chemiluminescent labels. When radioisotope labels are employed, radiometric scanning or reading can be used to detect the signal emitted from the hybridized features. Additional types of signals are also possible, including electrical signals generated by electrical properties of bound target molecules, magnetic properties of bound target molecules, and other such physical properties of bound target molecules that can produce a detectable signal. Optical, radiometric, or other types of scanning and reading produce an analog or digital representation of the array as shown in FIG. 7, with features to which labeled target molecules are hybridized similar to 706 optically or digitally differentiated from those features to which no labeled DNA molecules are bound. In other words, the analog or digital representation of a scanned array displays positive signals for features to which labeled DNA molecules are hybridized and displays negative features to which no, or an undetectably small number of, labeled DNA molecules are bound. Features displaying positive signals in the analog or digital representation indicate the presence of DNA molecules with complementary nucleotide sequences in the original sample solution. Moreover, the signal intensity produced by a feature is generally related to the amount of labeled DNA bound to the feature, in turn related to the concentration, in the sample to which the array was exposed, of labeled DNA complementary to the oligonucleotide within the feature.

One, two, or more than two data subsets within a data set can be obtained from a single molecular array by scanning or reading the molecular array for one, two or more than two types of signals. Two or more data subsets can also be obtained by combining data from two different arrays. When optical scanning or reading is used to detect fluorescent or chemiluminescent emission from chromophore labels, a first set of signals, or data subset, may be generated by scanning or reading the molecular array at a first optical wavelength, a second set of signals, or data subset, may be generated by scanning or reading the molecular array at a second optical wavelength, and additional sets of signals may be generated by scanning or reading the molecular at additional optical wavelengths. Different signals may be obtained from a molecular array by radiometric scanning or reading to detect radioactive emissions one, two, or more than two different energy levels. Target molecules may be labeled with either a first chromophore that emits light at a first wavelength, or a second chromophore that emits light at a second wavelength. Following hybridization, the molecular array can be scanned or read at the first wavelength to detect target molecules, labeled with the first chromophore, hybridized to features of the molecular array, and can then be scanned or read at the second wavelength to detect target molecules, labeled with the second chromophore, hybridized to the features of the molecular array. In one common molecular array system, the first chromophore emits light at a red visible-light wavelength, and the second chromophore emits light at a green, visible-light wavelength. The data set obtained from scanning or reading the molecular array at the red wavelength is referred to as the “red signal,” and the data set obtained from scanning or reading the molecular array at the green wavelength is referred to as the “green signal.” While it is common to use one or two different chromophores, it is possible to use one, three, four, or more than four different chromophores and to scan or read a molecular array at one, three, four, or more than four wavelengths to produce one, three, four, or more than four data sets.

As will be discussed below, current molecular array scanners employ laser/photo detector pairs, each pair designed to excite, and to detect signal from, a particular chromophore label. For example, current molecular array scanners designed for scanning molecular arrays exposed to target molecules labeled with red and green chromophore currently employ two laser/detector pairs. These laser/detector pairs are often referred to as channels. In turn, the green signal and red signal may be referred to as the green channel and the red channel, whether with respect to a molecular-array scanner or to a molecular array, and molecular arrays and molecular array scanners may be referred to as two-channel molecular arrays and two-channel molecular-array scanners, respectively, when containing two chromophore labels and two laser/detector pairs designed to detect signals from the two chromophore labels. The photo detectors are filtered in order to detect fluorescent emission from a particular chromophore label at the light wavelength characteristic for fluorescent emission from the chomophore label. However, fluorescent emission at a characteristic wavelength may not be directly proportional to the density, or concentration, of a chromophore label initially deposited within a feature of a molecular array. For example, when target molecules labeled with a particular type of chromophore are adsorbed or bound to a surface at a sufficiently high density, the chromophores may interact with one another in ways that alter their fluorescent emission spectrum or inhibit fluorescent emission. Inhibition may occur, as self quenching, when energy, released when electrons within the chromophore fall from excited states back to minimum-energy states, is dissipated in ways other than fluorescent emission, including release as increase vibrational or rotational energy in chromophore dimers or aggregates, or in radiation release at different wavelengths. As another example, a chromophore label may decompose, over time, by various reaction mechanisms into one or more decomposition products that do not absorb light at the frequency produced by the laser designed to excite the original chromophore and do not therefore emit fluorescent light in response to illumination by the laser light within the molecular array scanner, or absorb light with a different efficiency and fluoresce with a different characteristic fluorescent-emission spectrum. Various different types of interactions may, in addition, arise between two or more different types of chromophores deposited on the surface of the molecular array, altering their absorption or emission spectra. All of these phenomena may alter the relationship between the intensity of laser light impinging on the surface of the molecular array during a scan, the density of concentration of chromophore label deposited on the surface of the molecular array, the intensity of fluorescent emission at the characteristic wavelength for the chromophore. Currently, the effects of these phenomena are not directly measured by molecular array scanners, and are instead estimated, approximated, or extrapolated with significant inherent uncertainties and deviations. For these reasons, designers, manufactures, and users of molecular array scanner recognize the need for more accurately scanning the features of a molecular array to directly account for various phenomena that may alter the proportionality between the density, or concentration, or chormophore label initially deposited within a feature of a molecular array and the intensity of fluorescent emission measured by the molecular scanner from the feature at a later time.

### SUMMARY OF THE INVENTION

One embodiment of the present invention provides a method and system for reading the features of a molecular array in order to correct for fluorescent-emission spectral changes, such as those from chemical decomposition and other phenomena related to a chromophore label that can alter the relationship between the density, or concentration, of chromophore label initially present within a feature of a molecular array and the intensity of light emitted from the feature at a characteristic wavelength. Common, currently available molecular array scanners employ two laser/photo detectors pairs in order to scan for, and detect fluorescent emission from two different chromophore labels. Currently, molecular array scanners with four detectors are available, and, in the future, molecular array scanners with more than four detectors may be commonly available for scanning the features of a molecular array to detect the presence of more than four different chromophore labels. While current molecular array experiments generally involve two sample solutions and two different chromophore labels, four-channel molecular array scanners provide a means for scanning four-channel molecular arrays. These four-channel molecular arrays are intended to enable experiments using four sample solutions, and thus, for example, allow for analysis of gene expression at four different time points, or in four different tissues, rather than the two-time-point or two-tissue experiments currently possible with two-channel molecular arrays. However, four-channel molecular array scanners can be used, in addition, to read features of two-channel molecular arrays. Currently, only two of the four detectors of a four-channel molecular array scanner are intended to be used for scanning two-channel molecular arrays. However, with proper tuning and filtering, all four detectors of a four-channel molecular array scanner can be employed to measure signal from a two-channel array. The additional two channels may be used to directly measure signals at wavelengths at which spectral weight is shifted due to various causes. These causes may include chromophore decomposition products on the surface of the molecule array, various self-quenching or density-related, spectral signal-altering phenomena, peak broadening, peak shifting, or other phenomena that alter the relationship between the initial amount, density, or concentration, of a chromophore label and the fluorescent emission later detected by a molecular array scanner at a particular wavelength. By using four different signals, rather than two signals, to scan a two-channel molecular array, a more accurate determination of the initial concentration, or density, of each of two chromophore labels present within features of the molecular array can be determined. The method of the present invention can be applied, as well, to four-channel molecular arrays scanned in eight-channel molecular array scanners, or to any molecular array scanner with the capability of measuring a greater number of signals than the types of labels applied to the molecular array. For each additional signal above a number of signals equivalent to the number of channels of the molecular array, the instrument can detect, disambiguate, or deconvolve one additional spectral signal from those of the primary signals corresponding to originally applied labels.

### BRIEF DESCRIPTION OF THE DRAWINGS

FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108.

FIGS. 2A-B illustrate the hydrogen bonding between the purine and pyrimidine bases of two anti-parallel DNA strands.

FIG. 3 illustrates a short section of a DNA double helix 300 comprising a first strand 302 and a second, anti-parallel strand 304.

FIGS. 4-7 illustrate the principle of the array-based hybridization assay.

FIG. 8 is a block diagram of major optical and electronic components of a molecular array scanner.

FIG. 9 shows an abstract representation of the excitation and emission-detection components of a molecular array scanner.

FIG. 10 shows the spectrum of fluorescent emission from a first chromophore label C1.

FIG. 11 shows the spectrum of fluorescent emission from a second chromophore label C2 in the hypothetical example.

FIG. 12 shows a hypothetical spectrum of fluorescent emission from equal amounts of chromophore C1 and C2 adsorbed to a surface or in a solution.

FIG. 13 shows a hypothetical spectrum of fluorescent emission from a solution or surface, initially containing equal amounts of chromophore C1 and C2, after decomposition of 20% of chromophore C2 to chromophore C2_{d.}

FIG. 14 shows the spectrum of fluorescent emission from the pure decomposition product, C2_{d}, of the chromophore label C2.

FIG. 15 shows a hypothetical spectrum of fluorescent emission from a solution or surface, initially containing equal amounts of chromophore C1 and C2, after decomposition of 50% of chromophore C2 to chromophore C2_{d}.

FIG. 16 shows a spectrum of fluorescent emission from a solution or surface containing a concentration of chromophore C1 four times more than the concentration of chromophore C2.

FIG. 17 shows a spectrum of fluorescent emission from a solution or surface, initially containing a concentration of chromophore C1 6.67 times more than the concentration of chromophore C2, after decomposition of 20% of chromophore C2 to chromophore C2_{d}.

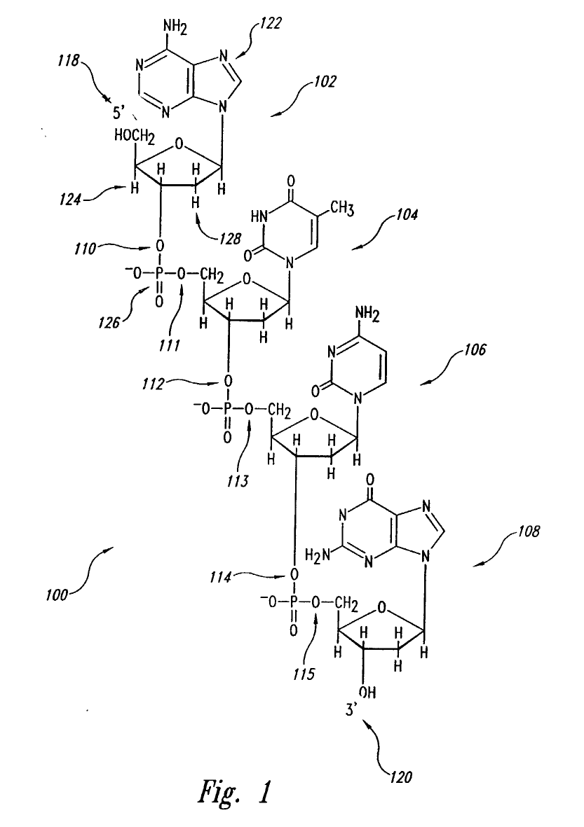

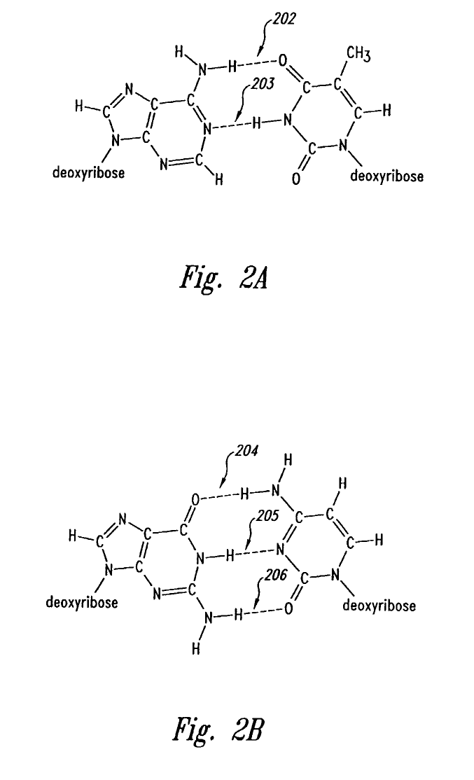

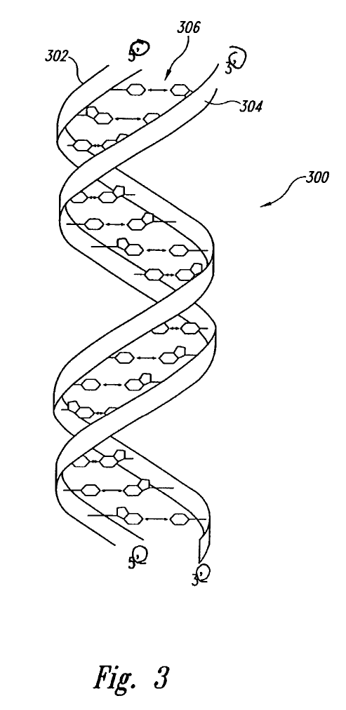

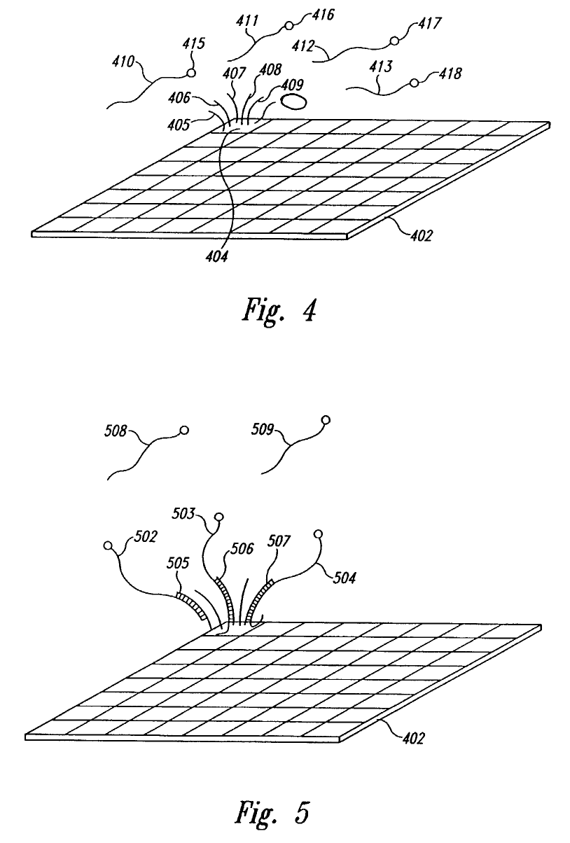

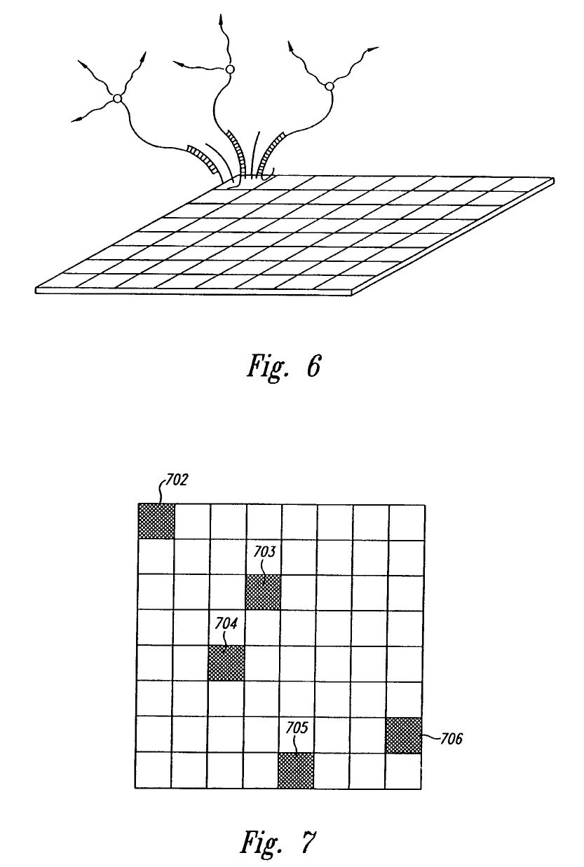

### DETAILED DESCRIPTION OF THE INVENTION

One embodiment of the present invention provides a means for detecting and using a greater number of signals than the number of channels of a molecular array to more accurately determine the initial densities, or concentrations, of chromophore labels applied to features of a molecular array. In a first subsection, below, additional information about molecular arrays in provided. In a second subsection, below, additional information about molecular array scanners is provided. Finally, in a third subsection, the method and system of one embodiment of the present invention is described with reference to FIGS. 10-17.

### Additional Information About Molecular Arrays

An array may include any one-, two- or three-dimensional arrangement of addressable regions, or features, each bearing a particular chemical moiety or moieties, such as biopolymers, associated with that region. Any given array substrate may carry one, two, or four or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm or even less than 10 cm². For example, square features may have widths, or round feature may have diameters, in the range from a 10 μm to 1.0 cm. In other embodiments each feature may have a width or diameter in the range of 1.0 μm to 1.0 mm, usually 5.0 μm to 500 μm, and more usually 10 μm to 200 μm. Features other than round or square may have area ranges equivalent to that of circular features with the foregoing diameter ranges. At least some, or all, of the features may be of different compositions (for example, when any repeats of each feature composition are excluded the remaining features may account for at least 5%, 10%, or 20% of the total number of features). Interfeature areas are typically, but not necessarily, present. Interfeature areas generally do not carry probe molecules. Such interfeature areas typically are present where the arrays are formed by processes involving drop deposition of reagents, but may not be present when, for example, photolithographic array fabrication processes are used. When present, interfeature areas can be of various sizes and configurations.

Each array may cover an area of less than 100 cm², or even less than 50 cm², 10 cm² or 1 cm². In many embodiments, the substrate carrying the one or more arrays will be shaped generally as a rectangular solid having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. Other shapes are possible, as well. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein. Also, instead of drop deposition methods, photolithographic array fabrication methods may be used such as described in U.S. Pat. No. 5,599,695, U.S. Pat. No. 5,753,788, and U.S. Pat. No. 6,329,143. Interfeature areas need not be present particularly when the arrays are made by photolithographic methods as described in those patents.

A molecular array is typically exposed to a sample including labeled target molecules, or, as mentioned above, to a sample including unlabeled target molecules followed by exposure to labeled molecules that bind to unlabeled target molecules bound to the array, and the array is then read. Reading of the array may be accomplished by illuminating the array and reading the location and intensity of resulting fluorescence at multiple regions on each feature of the array. For example, a scanner may be used for this purpose, which is similar to the AGILENT MICROARRAY SCANNER manufactured by Agilent Technologies, Palo Alto, Calif. Other suitable apparatus and methods are described in U.S. patent application Ser. No. 10/087,447 “Reading Dry Chemical Arrays Through The Substrate” by Corson et al., and Ser. No. 09/846,125 “Reading Multi-Featured Arrays” by Dorsel et al. However, arrays may be read by any other method or apparatus than the foregoing, with other reading methods including other optical techniques, such as detecting chemiluminescent or electroluminescent labels, or electrical techniques, for where each feature is provided with an electrode to detect hybridization at that feature in a manner disclosed in U.S. Pat. No. 6,251,685, U.S. Pat. No. 6,221,583 and elsewhere.

A result obtained from reading an array may be used in that form or may be further processed to generate a result such as that obtained by forming conclusions based on the pattern read from the array, such as whether or not a particular target sequence may have been present in the sample, or whether or not a pattern indicates a particular condition of an organism from which the sample came. A result of the reading, whether further processed or not, may be forwarded, such as by communication, to a remote location if desired, and received there for further use, such as for further processing. When one item is indicated as being remote from another, this is referenced that the two items are at least in different buildings, and may be at least one mile, ten miles, or at least one hundred miles apart. Communicating information references transmitting the data representing that information as electrical signals over a suitable communication channel, for example, over a private or public network. Forwarding an item refers to any means of getting the item from one location to the next, whether by physically transporting that item or, in the case of data, physically transporting a medium carrying the data or communicating the data.

As pointed out above, array-based assays can involve other types of biopolymers, synthetic polymers, and other types of chemical entities. A biopolymer is a polymer of one or more types of repeating units. Biopolymers are typically found in biological systems and particularly include polysaccharides, peptides, and polynucleotides, as well as their analogs such as those compounds composed of, or containing, amino acid analogs or non-amino-acid groups, or nucleotide analogs or non-nucleotide groups. This includes polynucleotides in which the conventional backbone has been replaced with a non-naturally occurring or synthetic backbone, and nucleic acids, or synthetic or naturally occurring nucleic-acid analogs, in which one or more of the conventional bases has been replaced with a natural or synthetic group capable of participating in Watson-Crick-type hydrogen bonding interactions. Polynucleotides include single or multiple-stranded configurations, where one or more of the strands may or may not be completely aligned with another. For example, a biopolymer includes DNA, RNA, oligonucleotides, and PNA and other polynucleotides as described in U.S. Pat. No. 5,948,902 and references cited therein, regardless of the source. An oligonucleotide is a nucleotide multimer of about 10 to 100 nucleotides in length, while a polynucleotide includes a nucleotide multimer having any number of nucleotides.

As an example of a non-nucleic-acid-based molecular array, protein antibodies may be attached to features of the array that would bind to soluble labeled antigens in a sample solution. Many other types of chemical assays may be facilitated by array technologies. For example, polysaccharides, glycoproteins, synthetic copolymers, including block copolymers, biopolymer-like polymers with synthetic or derivitized monomers or monomer linkages, and many other types of chemical or biochemical entities may serve as probe and target molecules for array-based analysis. A fundamental principle upon which arrays are based is that of specific recognition, by probe molecules affixed to the array, of target molecules, whether by sequence-mediated binding affinities, binding affinities based on conformational or topological properties of probe and target molecules, or binding affinities based on spatial distribution of electrical charge on the surfaces of target and probe molecules.

Scanning of a molecular array by an optical scanning device or radiometric scanning device generally produces a scanned image comprising a rectilinear grid of pixels, with each pixel having a corresponding signal intensity. These signal intensities are processed by an array-data-processing program that analyzes data scanned from an array to produce experimental or diagnostic results which are stored in a computer-readable medium, transferred to an intercommunicating entity via electronic signals, printed in a human-readable format, or otherwise made available for further use. Molecular array experiments can indicate precise gene-expression responses of organisms to drugs, other chemical and biological substances, environmental factors, and other effects. Molecular array experiments can also be used to diagnose disease, for gene sequencing, and for analytical chemistry. Processing of molecular-array data can produce detailed chemical and biological analyses, disease diagnoses, and other information that can be stored in a computer-readable medium, transferred to an intercommunicating entity via electronic signals, printed in a human-readable format, or otherwise made available for further use.

### Additional Information About Molecular Array Scanners

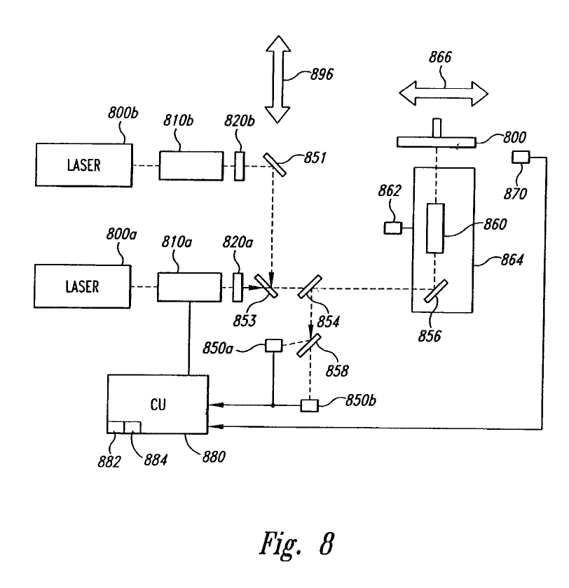

FIG. 8 illustrates components of a molecular array scanner. Lasers 800a-b emit coherent light that passes through electro-optic modulators (“EOMs”) 810a-b with attached polarizers 820a-b. Each EOM and corresponding polarizer together act as a variable optical attenuator. A control signal in the form of a variable voltage is applied to each EOM 810a-b by controller 880. The controller 880 may include a suitably programmed processor, logic circuit, firmware, or a combination of software programs, logic circuits, and firmware. The control signal changes the polarization of the laser light, which alters the intensity of the light that passes through the EOM. In general, laser 800a provides coherent light of a different wavelength than that provided by laser 810b. For example, one laser may provide red light and the other laser may provide green light. The beams may be combined along a path toward a stage 800 by the use of full mirror 851 and dichroic mirror 853. The light from the lasers 800a-b is then transmitted through a dichroic beam splitter 854, reflected off fully reflecting mirror 856, and then focused, using optical components in beam focuser 860, onto a molecular array mounted on a holder 800. Fluorescent light, emitted at two different wavelengths (for example, green light and red light) from features of the molecular array in response to illumination by the laser light, is imaged using the optics in the focuser/scanner 860, and is reflected off mirrors 856 and 854. The distinct excitation sources are aligned such that the emitted fluorescence passes through a further dichroic mirror 858 and are passed to respective detectors 850a and 850b. More optical components (not shown in FIG. 8) may be used between the dichroic mirror and the photodetectors 850a-b, such as lenses, pinholes, filters, and fibers. The photodetectors 850a-b may be of various different types, including photo-multiplier tubes, charge-coupled devices, and avalanche photodiodes.

A scan system causes a light spot from each laser 800a-b to be moved in a regular pattern about the surface of the molecular array. The molecular array is mounted to a stage that can be moved in horizontal and vertical directions to position light from the lasers onto a particular region at the surface of the molecular array, from which region fluorescent emission is passed back to the photodetectors via the optical path described above. An autofocus detector 870 is provided to sense and correct any offset between different regions of the molecular array and the focal plane of the system during scanning. An autofocus system includes detector 870, processor 880, and a motorized adjuster to move the stage in the direction of arrow 896.

The controller 880 receives signals from photodetectors 850a-b, called “channels,” corresponding to the intensity of the green and red fluorescent light emitted by probe labels excited by the laser light. The controller 880 also receives a signal from autofocus offset detector 870 in order to control stage adjustment, provides the control signal to the EOMs 810a-b, and controls the scan system. Controller 880 may also analyze, store, and output data relating to emitted signals received from detectors 850a-b.

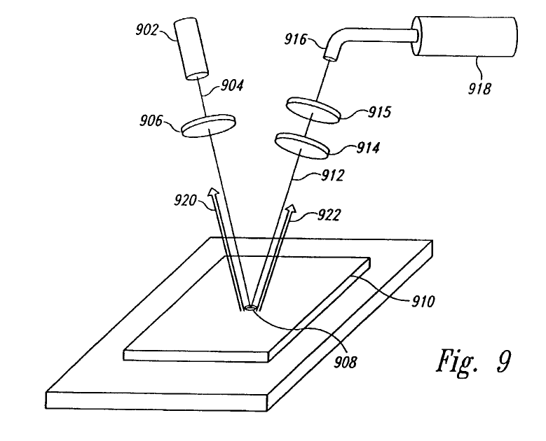

FIG. 9 shows an abstract representation of the excitation and emission-detection components of a molecular array scanner. In FIG. 9, a laser source 902 generates a coherent light beam of a particular wavelength 904 that may be optically filtered and focused by filtering and focusing components 906 to impinge on a small region 908 of the surface of a molecular array 910. Excited target molecules hybridized to the surface of the molecular array are excited by the impinging laser light and subsequently emit fluorescent light, generally at a lower wavelength. The emitted light 912 is focused and filtered by various focusing and filtering components 914-915 to impinge on an optical fiber 916, or another type of light collection material, and input into a photodetector 918 to generate an electronic signal proportional to the intensity of the emitted light falling onto the optical fiber 916. It may be possible to include electronic and optical feedback mechanisms, represented in FIG. 9 by arrows 920 and 922, to ensure steady and constant laser illumination intensity at the surface of the molecular array and a steady output voltage following conversion of the current signal output from the photodetector for a known emitted light energy level of a particular wavelength. However, molecular array scanners do not contain a calibration mechanism for correlating the intensity of the laser light to the intensity of the emitted light from the surface of the molecular array. Correlation between the signal produced by the photodetector and the light energy generated by the laser involves many different components of the molecular array scanner and a molecular array being scanned. For example, differences in the filtering and optical components in different molecular array scanners may cause differences in the signals generated by the molecular array scanners with respect to constant laser-light intensity. As another example, differences in the surface characteristics of a molecular array, including differences in the chemical environment of fluorophores or chromophores employed to label probe molecules, can result in variations in the signal generated for a given laser-light intensity. In general, the relationship between laser-light intensity and the electronic signal corresponding to emitted-light intensity from probe labels must be determined by a calibration method for each molecular array scanner. Molecular array scanners of a particular type also need to be calibrated amongst themselves, so that scanning of a standard reference array generates the same electronic output signal in every molecular array scanner of the particular type.

### One Embodiment of the Present Invention

Currently, 2-channel molecular array scanners, as discussed above with reference to FIG. 8, are most commonly encountered, and at least one 4-channel molecular array scanner is commercially available. Future molecular array scanners may employ more than four detectors. In certain cases, additional detectors with different spectral responses may be used without increasing the number of lasers. For example, it may be possible to build a four-channel molecular array scanner using only two lasers and four photo detectors. Chromophore labels 1 and 2 may both be excited by the wavelength of light produced by a first laser, and the chromophore labels 3 and 4 may be excited by the wavelength of light produced by a second laser. Four photo detectors with four different spectral responses may then be employed to detect the four different characteristic wavelengths of light fluorescently emitted by the four different chromophore labels. Alternatively, rather than employ additional photo detectors, dynamic filter adjustments may be possible to enable a single detector to be used to detect fluorescent emission at two or more different wavelengths. In other cases, it may be necessary to include four different laser/detector pairs. However, in all cases, a four-channel molecular array scanner is able to scan a molecular array for fluorescent emission at four different wavelengths of light, or within four different wavelength regions, or bands.

While a four-channel molecular array scanner is needed to process a four-channel molecular array, as discussed above, a four-channel molecular array scanner may also be used to process a two-channel molecular array. In the latter case, rather than using only two of the four signals scanned by the molecular array scanner to process the two-channel molecular array, one embodiment of the present invention employs all four signals to more accurately determine the initial densities, or concentrations, of the two chromophore labels within features of a two-channel molecular array. The concept of the present invention may be extended to any case where a greater number of signals can be detected by a molecular array scanner than the number of chromophore labels deposited on the surface of the molecular array scanned by the molecular array scanner.

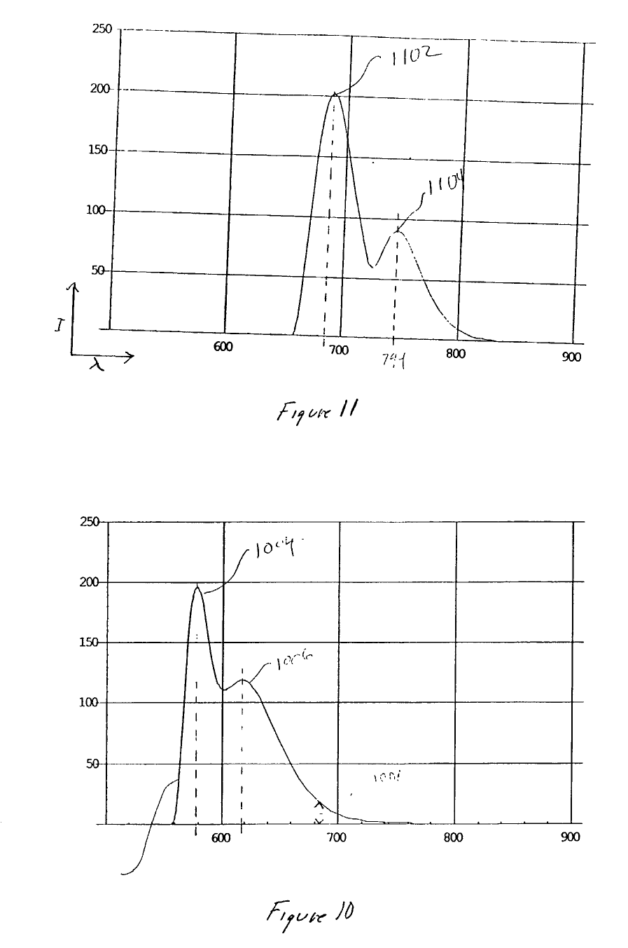

FIG. 10 shows the hypothetical spectrum of fluorescent emission from a first chromophore label C1. The graphical representation technique used in FIG. 10 is also employed in FIGS. 11-17, discussed below. In all of these figures, the horizontal axis 1002 represents the wavelength λ of the fluorescent light emitted from the surface of the molecular array, and the vertical axis 1004 represents the intensity I of the fluorescent light. The vertical axis is arbitrarily scaled for the hypothetical example. In FIG. 10, the fluorescent-emission spectrum 1002 from C1 can be seen to comprise a major peak 1004 and a minor peak 1006 at wavelengths 578 nm (“λ₅₇₈”) and 618 nm (“λ₆₁₈”), respectively. FIG. 10 represents spectrum that might be obtained using a tunable scanning device that measures the intensity of fluorescent emission over a range of wavelengths, and excites the chromophore using a broad range of wavelengths. In a molecular array scanner, by contrast, a very small, sharply focused, monochromatic excitation beam is employed to image the surface of a molecular array, and detectors are generally filtered to detect fluorescent light within a narrow wavelength range. Therefore, a molecular array scanner cannot detect or measure a spectrum, such as the spectra shown in FIGS. 10-17, but instead detects the intensity of fluorescent emission within a narrow wavelength range from a tiny portion of the surface of a molecular array. FIGS. 10-17 show spectral curves to illustrate appropriate choices for narrow wavelength windows, measured by detectors of a molecular array scanner that can be used to infer label concentrations.

FIG. 11 shows a hypothetical spectrum of fluorescent-emission from a second chromophore label C2. It can be seen in FIG. 11 that chromophore C2 has a major fluorescent-emission peak 1102 at λ₆₈₄ and a minor peak 1104 at λ₇₄₄.

FIG. 12 shows a hypothetical spectrum of fluorescent emission from equal amounts of chromophore C1 and C2 adsorbed to a surface or in a solution. The two major peaks in this combined spectrum 1202 and 1204 correspond to the major C1 peak at λ₅₇₈ and the major C2 peak at λ₄₈₄. By examining the combined spectrum, the choice of detectors sensitive to wavelengths λ₅₇₈ and λ₆₈₄ for scanning two-channel molecular arrays employing chromophore labels C1 and C2 is straightforward. The measured intensities at wavelengths λ₅₇₈ and λ₄₈₄ are generally, over a wide range of intensity values, proportional to the densities, or concentrations, of chromophore labels C1 and C2, respectively. Note, however, that signal from both C1 and C2 contribute to the intensity of light emitted from the surface at λ₆₈₄. Whereas, in the spectrum from pure C2, shown in FIG. 11, the measured intensity at the peak is 200, the same density, or concentration of C2 gives rise to a λ₆₈₄ peak intensity of 220, due to contribution from the overlapping C1 signal. The overlap can be seen, in FIG. 10, as the vertical height of the curve of the C1 chromophore 1008 at 684 nm.

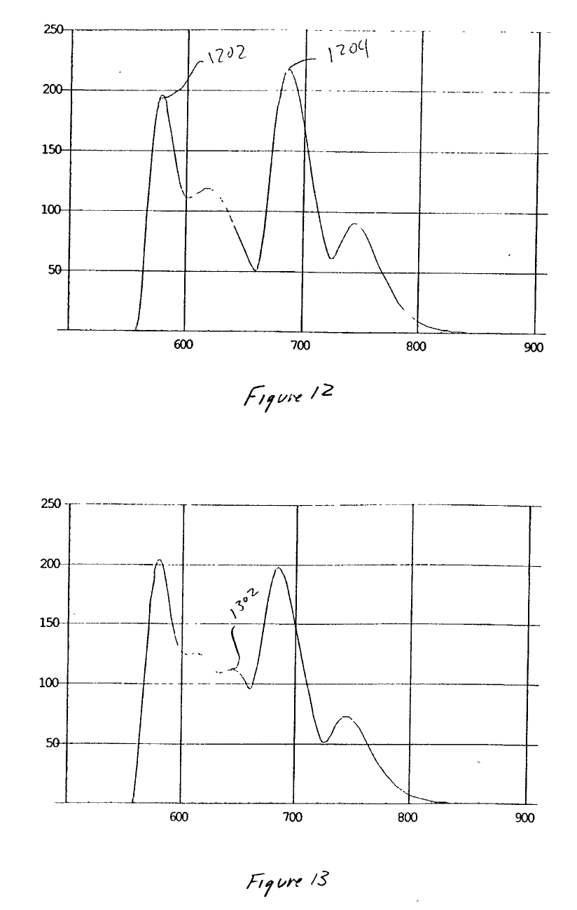

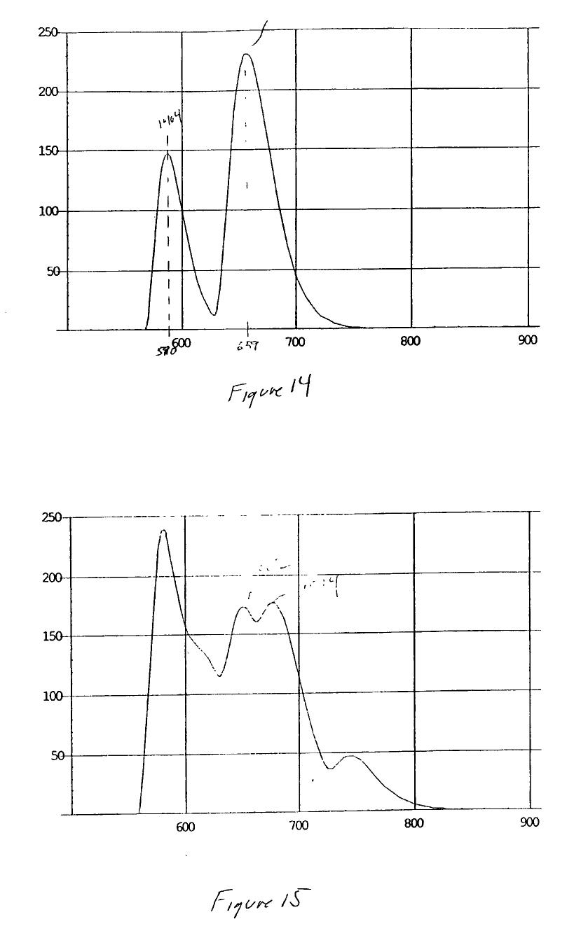

However, consider the spectrum shown in FIG. 13. FIG. 13 shows a hypothetical spectrum of fluorescent emission from a solution or surface, initially containing equal amounts of chromophore C1 and C2, after decomposition of 20% of chromophore C2 to chromophore C2_{d}. A new peak 1302, corresponding to decomposition product C2_{d}, is seen in the spectrum shown in FIG. 13. Note that the intensity of the characteristic C2 peak at λ₆₈₄ has decreased, relative to the intensity of the characteristic C1 peak at λ₅₇₈, in contrast to the spectrum shown in FIG. 12. Thus, decomposition of C2 to C2_{d} results in a new peak and a corresponding decrease in C2 684 intensity. FIG. 14 shows the spectrum of fluorescent emission from the pure decomposition product, C2_{d}, of the chromophore label C2. The pure spectrum shows C2_{d} peaks at λ₅₉₀ and λ₆₅₉. Thus, the presence of C2 decomposition product C2_{d} contributes significant added signal to the characteristic C1 peak at λ₅₇₈ as well as to the characteristic C2 peak at λ₆₈₄.

Decomposition of C2 to C2_{d} may occur prior to application of C2-labeled target molecules to a molecular array, following application of C2-labeled target molecules to a molecular array, or both. In terms of chromophore density measurement, however, the effect in both cases results in a lower measured density, or concentration of C2, than the amount of C2-labeled and C2_{d}-labeled target molecules on the surface when the density determination is based on measured signal intensities at wavelengths of the C1 and C2 peak intensities, rather than the C2_{d} peak intensities.

The two channels of a two-channel molecular array generally correspond, in gene expression experiments, to expression levels of genes at two different times or two different tissues. It is the relative gene-expression levels that an experimenter hopes to determine in order to correlate observed phenomena with gene-expression levels. The gene-expression levels, as reflected in the concentration of cDNA copies of gene mRNA transcription products in sample solutions, is directly proportional to the amount of C1 and C2 chromophores initially present within features of the molecular array. Thus, in scanning a molecular array, the experimenter hopes to obtain signal intensities directly proportional to the initial C1 and C2 concentrations. However, as shown in the above example, due to decomposition of chromophore C2 to C2_{d}, the measured signal intensity at λ₆₈₄ is not directly proportional to the amount of C2-labeled or C2_{d}-labeled target molecules adsorbed to, or bound to, the surface, nor is the measured signal intensity at λ₅₇₈ directly proportional to the C1 concentration. The experimenter hopes to measure the density of the total amount of target molecule labeled with C2 or C2_{d}, or, in other words, the density or concentration of C2_{T}-labeled target molecule, but, in a two channel molecular array scanner, the experimenter can assume only the he or she is measuring the density or concentration of C2-labeled target molecule. C2_{T} is the sum of the concentrations, or densities, of C2 and C2_{d}. Thus, in the situation shown in FIG. 13, although the C1/C2_{T} ratio is 1.0, the measured C1^{/C}2 ratio is 1.25. The results of the molecular array scan would thus indicate over-expression of the C1-labeled gene product with respect to the C2-labeled gene product due to decomposition of C2 and failure to measure both C2 and C2_{d}, when, in fact, both genes are expressed at the same level.

FIG. 15 shows a hypothetical spectrum of fluorescent emission from a solution or surface, initially containing equal amounts of chromophore C1 and C2, after decomposition of 50% of chromophore C2 to chromophore C2_{d}. In FIG. 15, although the C1/C2_{T} ratio is 1.0, the measured C1/C2 ratio is nearly 1.5. In this case, a failure to take into account and measure the decomposition of C2 would lead to a dramatically false result.

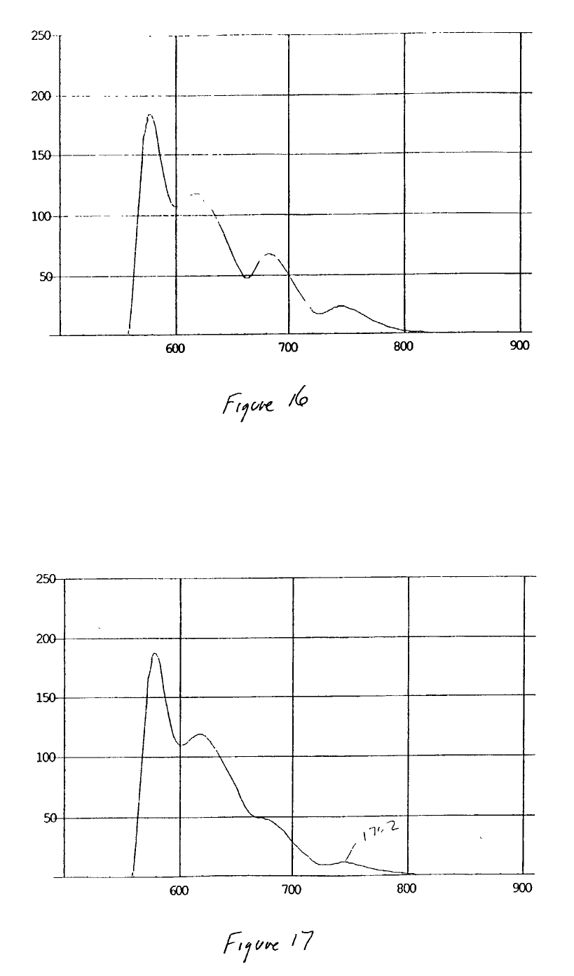

FIG. 16 shows a spectrum of fluorescent emission from a solution or surface containing a concentration of chromophore C1 four times more than the concentration of chromophore C2. As the concentration, or density, of C2 decreases relative to C1, the spectral curve for C2 begins to be overshadowed by that of C1. FIG. 17 shows a spectrum of fluorescent emission from a solution or surface, initially containing a concentration of chromophore C1 6.67 times more than the concentration of chromophore C2, after decomposition of 20% of chromophore C2 to chromophore C2_{d}. At this relative difference in concentrations of C2 and C1, the major C2 peak at λ₆₈₄ is no longer distinctly visible in the combined spectrum. But, more seriously, the magnitude of the C2 intensity contribution to the measured intensity at λ₆₈₄ may be smaller than the magnitude of the expected error in intensity at λ₆₈₄ contributed by C1. In other words, the noise inherent in the intensity contribution due to the C1 spectral overlap at λ₆₈₄ (1008 in FIG. 10) may completely mask the relatively tiny signal due to C2. When a significant amount of C2 has decomposed to C2_{d}, overlap from the C2_{d} signal may further complicate the task of identifying the C2 signal.

Thus, FIGS. 13-17 have illustrated two related problems with the common two-channel-molecular-array-scanner approach to reading data from a two-channel molecular array. First, decomposition of one or both chromophores can lead to a failure to detect a significant portion of target molecules adsorbed to, or bound to, a surface, that were initially labeled with a particular chromophore, but that, following decomposition of that chromophore, are labeled with a decomposition product that produces either no detectable signal, or a signal at a different wavelength from the detected wavelength. A second problem relates to signal overlap, and the masking of a weak signal from a chromophore by the variance, or noise, in an overlapping signal from a second chromophore. Many other similar problems may occur. For example, the width of a peak may broaden, due to interaction between chromophore molecules at high density. Broadening of a peak changes the proportionality between peak height and chromophore density, as the density is more directly related to the area underneath the spectral curve.

In all of these cases, the problem essentially amounts to an inability to detect spectral changes, due to any of various causes, in the two, narrow windows into the spectrum provided by two detectors. The two detectors allow, for example, the intensities of two characteristic peak wavelengths to be determined, but do not allow for detection of a decomposition product, masking by a much stronger, overlapping signal, or changes in the spectrum, such as peak broadening.

One embodiment of the present invention involves using surplus channels in a molecular array scanner as additional windows into the spectrum, to aid in detecting the spectrum-altering phenomena described above. For example, consider the C1/C2 system described above with reference to FIGS. 10-17. If a third detector can be tuned to detect signal at λ₄₅₉, the peak intensity of the C2_{d} decomposition product (1402 in FIG. 14), then the density, or concentration of C2_{d} can be determined as a function of the measured signals. If a fourth detector can be tuned to the wavelength of the minor C2 peak (1104 in FIG. 11), λ₇₄₄, then a second direct C2 signal is available. This second C2 signal is not appreciably overlapped by the signal from either C1 or C2_{d}, and is still visible 1702 in the combined spectrum shown in FIG. 17. So even when the major peak is masked by noise from a much stronger C1 signal, the minor peak at λ₇₄₄ can be used to quantify the C2 signal.

In particular, the following matrix equation expresses the relationship between the measured intensities at λ₅₇₈, λ₆₈₄, and λ₆₅₉ and the concentrations, or densities, of C1, C2, and C2_{d}:

$$[ \begin{matrix}97.5 & 0 & 37 \\ 17.5 & 101.5 & 51 \\ 26 & 0 & 180\end{matrix} ] [ \begin{matrix}C 1 \\ C 2 \\ C {2}_{d}\end{matrix} ] = [ \begin{matrix}{D}_{λ 578} \\ {D}_{λ 684} \\ {D}_{λ 659}\end{matrix} ]$$

Note that the concentrations of all three of C1, C2, and C2_{d} contribute to the measured signal at λ₄₈₄, the concentrations of C1 and C2_{d} contribute to the measured signal at λ_{659,} and only the concentration of C1 contributes to the measured signal at λ₅₇₈. In many, non-hypothetical two-channel molecular arrays, all three of C1, C2, and C2_{d} contribute to all three measured intensities at λ₅₇₈, λ₄₈₄, and λ₆₅₉, and there are no 0 elements in the 3×3 matrix. Inversion of the 3×3 matrix provides:

$$[ \begin{matrix}C1 \\ C2 \\ {C2}_{d}\end{matrix} ] = [ \begin{matrix}0.0109 & 0 & - 0.0022 \\ - 0.0011 & 0.0099 & - 0.0026 \\ - 0.0016 & 0 & 0.0059\end{matrix} ] [ \begin{matrix}{D}_{λ 578} \\ {D}_{λ 684} \\ {D}_{λ 659}\end{matrix} ]$$

This equation can be straightforwardly programmed into a molecular array scanner or molecular array data processing system in order to derive the concentrations of all three of C1, C2, and C2_{d} from the measured intensities at λ₅₇₈, λ₆₄₈, and λ₆₅₉.

When the magnitude of the C1 signal exceeds that of the C2 signal by a sufficient amount, or, in other words, when the ratio D_{λ578}/D_{λ684} exceeds some threshold value, such as 10.0, then, rather than using the intensities at λ₅₇₈, λ₆₈₄, and λ659 to determine the concentrations, or densities, of C1, C2, and C2_{d}, the measured intensities at λ₅₇₈, λ₇₄₄, and λ₆₅₉ can be instead employed, as follows:

$$\begin{matrix}[ \begin{matrix}97.5 & 0 & 37 \\ 0 & 43.5 & 0.5 \\ 26 & 0 & 180\end{matrix} ] [ \begin{matrix}C 1 \\ C 2 \\ C {2}_{d}\end{matrix} ] = [ \begin{matrix}{D}_{λ 578} \\ {D}_{λ 744} \\ {D}_{λ 659}\end{matrix} ] \\ [ \begin{matrix}C 1 \\ C 2 \\ C {2}_{d}\end{matrix} ] = [ \begin{matrix}0.0109 & 0 & - 0.0022 \\ 0 & 0.0230 & - 0.0001 \\ - 0.0016 & 0 & 0.0059\end{matrix} ] [ \begin{matrix}{D}_{λ 578} \\ {D}_{λ 744} \\ {D}_{λ 659}\end{matrix} ]\end{matrix}$$

By the above matrix equations, the measured intensity at the minor C2 peak wavelength λ₇₄₄ is used, rather than the measured intensity at the major C2 peak wavelength λ₆₈₄, to determine the concentration, or density, of C2 without overlap from the C1 and C2_{d} signals. Again, in non-hypothetical cases, there may be very small, but non-zero contributions of C1 and C2_{d} signals to the measured intensity at λ₆₅₉.

Alternatively, C2 can be computed in both ways, and an average or weighted average C2 based on both computations of C2 may be returned. In one embodiment of an array scanner, C_{T} may be calculated by the scanner, and reported, rather than C2 or C2_{d}, so that the scanner reports only a C1 and a C2 value for each pixel.

In certain cases, rather than measuring narrow wavelength regions, a molecular array detector may instead respond to fluorescent light having a broader range of wavelengths. The same general approach of the present invention nonetheless applies. By employing surplus scanner channels to extract more information from features of a molecular array, the molecular array scanner or molecular array data processing system can disambiguate and deconvolve signal overlaps and spectral changes resulting from chromophore interactions and decomposition. In the above discussion, the problem of peak broadening causing changes to the proportionality between peak height and chromophore density was noted. For such cases, an extra channel that detects the measured intensity at a wavelength corresponding to a shoulder of the major peak may provide sufficient information, along with the measured intensity at the major peak wavelength, to accurately account for peak broadening. In some cases, insufficient surplus channels may be available to collect sufficient information for an unambiguous and unique solution of the measure-signal/chromophore concentration equations, but the additional information gleaned from surplus channels may still provide a better estimation of the true concentrations.

The above discussion has focused on chromophore labels, but the present invention may also be applied to other type of labels. For example, if short-lived radioisotope labels are used, it may be possible to measure signals corresponding to radioactive emission from decay products, in order to more accurately estimate the initial concentrations of radiolabeled target molecules.

It should be noted that the above hypothetical example shows only one of many different, possible spectral phenomena that leads a two-channel measurement to report chromophore concentrations, or densities, significantly different from the actual chromophore densities present on the surface of a molecular array. For example, there may be a C1 decomposition product, or numerous C1 and C2 decomposition products. Chromophore interactions may broaden or shift peaks. As long as the nature and spectral effects of these phenomena are known, measurement of additional signals can lead to direct determination, in some cases, and better estimates, on other cases, of the actual chromophore concentrations of densities on the surface of a molecular array based on measured signals.

In the future, it may be possible to dynamically adjust detector sensitivity and pre-filtering within a molecular array scanner in order to scan for fluorescent emission over a range of wavelengths. In these cases, the additional information obtained may be equivalent to scanning for a large number of signals with respect to the current four-channel molecular array scanners. This additional information can be used, according to the present invention, to more accurately determine the initial concentrations, or densities, of chromophore resident within features of the molecular array.

Although the present invention has been described in terms of a particular embodiment, it is not intended that the invention be limited to this embodiment. Modifications within the spirit of the invention will be apparent to those skilled in the art. For example, as previously discussed, any number of channels within a molecular array scanner greater than the number of channels of a molecular array can be used to more precisely determine initial concentrations of labels within features of the molecular array. The mathematical expressions relating measured signals to label concentrations vary with the types of labels and types of detectors. The above-described hypothetical example is one of an almost limitless number of possible signal/label-concentration relationship models. The mathematical expression or expressions for particular cases can be encoded into computer programs, logic circuits, or other control entities within a molecular array scanner or molecular-array data-processing system in order to derive initial label concentrations from measured signals. Each measured signal, or channel, produces a data subset, and the data subsets may be combined to produce a data set, which is then processed to provide initial concentrations, or densities, of labels within features of molecular arrays. For any particular case, an almost limitless number of different encodings and implementations are possible, depending on the particular hardware and software platforms used. The above discussion is directly related to molecular array data-acquisition devices that employ scanning methods in which narrow laser beams are used to scan across an array, region by region, and fluorescent emission from illuminated regions is detected and recorded as pixel intensities. The present invention may also be applied to molecular array data-acquisition devices that employ charge-coupled device arrays and other such area detectors in combination with filters to read the array by simultaneously acquiring characteristic fluorescent emission data from large regions of an array, or the entire array, at various pre-selected wavelengths.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for determining the amounts of a number of types of labels bound to a feature of a molecular array, the method comprising:
reading the feature to detect a characteristic signal for each of the number of types of labels bound to the feature as well as one or more additional signals; and
using the characteristic signals and the one or more additional signals to determine the amounts of the number of types of labels bound to a feature with greater accuracy than can be determined by using only the characteristic signals.

2. The method of claim 1 wherein each type of label is a molecule or chemical moiety that emits fluorescence.

3. The method of claim 1 wherein each type of label is a molecule or chemical moiety that includes a radioisotope.

4. The method of claim 1 wherein each characteristic signal corresponds to detected fluorescent emission of light at a particular wavelength or within a particular range of wavelengths.

5. The method of claim 1 wherein each characteristic signal corresponds to detected emission of radiation at a particular energy or within a particular range of energies.

6. The method of claim 1 wherein the one or more additional signals correspond to one or more different signals produced by one or more of the types of labels.

7. The method of claim 1 wherein the one or more additional signals correspond to a signal emitted by one or more decomposition products of one or more of the types of labels.

8. The method of claim 1 wherein reading the feature to detect one or more additional signals further includes reading the molecular array to detect fluorescent emission of light at a wavelength different from the wavelengths at which the characteristic signals are detected.

9. The method of claim 1 wherein reading the feature to detect one or more additional signals further includes reading the molecular array to detect emission of radiation at an energy different from the energies at which the characteristic signals are detected.

10. The method of claim 1 wherein a characteristic signal for a label and an additional signal corresponding to a decomposition product of the label are used together to determine the amount of the label initially bound to the molecular array.

11. The method of claim 1 wherein an additional signal produced by one or a combination of labels is used to resolve an unknown variable in a mathematical model of the relationship between measured signals and amounts of labels bound to the feature.

12. The method of claim 11 wherein the additional signal produced by one or a combination of labels corresponds to a minor peak in the emission spectrum of a particular chromophore that is not overlapped, or only insignificantly overlapped, by signal from other chromophores.

13. The method of claim 11 wherein the additional signal produced by one or a combination of labels corresponds to a shoulder of a major peak used, in addition a characteristic signal corresponding to the major peak, in order to accurately account for peak broadening or peak shifts due to chromophore-chromophore interactions.

14. The method of claim 11 wherein the additional signal produced by one or a combination of labels corresponds to a secondary emission from a molecule or chemical moiety excited by fluorescent emission from one or more of the number of types of labels.

15. Transferring results produced by a molecular array scanner or molecular array data processing program employing the method of claim 1 stored in a computer-readable medium to an intercommunicating entity.

16. Transferring results produced by a molecular array scanner or molecular array data processing program employing the method of claim 1 to an intercommunicating entity via electronic signals.

17. A computer program including an implementation of the method of claim 1 stored in a computer-readable medium.

18. A method comprising forwarding data produced by using the method of claim 1.

19. A method comprising receiving data produced by using the method of claim 1.

20. A molecular array scanner that employs the method of claim 1 to determine the amounts of a number of types of labels bound to features of the molecular array.

21. A molecular-array-data processing system comprising:
a computer processor;
a communications medium by which molecular array data are received by the molecular-array-data processing system;
one or more memory components that store molecular array data; and
a program, stored in the one or more memory components and executed by the computer processor, that determines the amounts of a number of types of labels bound to features of a molecular array by:
receiving molecular array data including data representing a characteristic signal read from the molecular array for each of a number of types of labels bound to the features of the molecular array as well as data representing one or more additional signals read from the molecular array; and
using the characteristic signals and the one or more additional signals to determine the amounts of the number of types of labels bound to the features with greater accuracy than can be determined by using only the characteristic signals.

22. The system of claim 1 wherein each type of label is a molecule or chemical moiety that emits fluorescence.

23. The system of claim 1 wherein each type of label is a molecule or chemical moiety that includes a radioisotope.

24. The system of claim 1 wherein each characteristic signal read from the molecular array corresponds to detected fluorescent emission of light at a particular wavelength or within a particular range of wavelengths.

25. The system of claim 1 wherein each characteristic signal read from the molecular array corresponds to detected emission of radiation at a particular energy or within a particular range of energies.

26. The system of claim 1 wherein the one or more additional signals read from the molecular array correspond to one or more different signals produced by one or more of the types of labels.

27. The system of claim 1 wherein the one or more additional signals read from the molecular array correspond to a signal emitted by one or more decomposition products of one or more of the types of labels.

28. The system of claim 1 wherein the one or more additional signals read from the molecular array correspond to fluorescent emission of light at one or more wavelengths different from the wavelengths at which the characteristic signals are detected.

29. The system of claim 1 wherein the one or more additional signals read from the molecular array correspond to emission of radiation at an energy or energies different from the energies at which the characteristic signals are detected.

30. The system of claim 1 wherein a characteristic signal for a label and an additional signal corresponding to a decomposition product of the label are used together to determine the amount of the label initially bound to the molecular array.

31. The system of claim 1 wherein an additional signal produced by one or a combination of labels is used to resolve an unknown variable in a mathematical model of the relationship between measured signals and amounts of labels bound to the feature.

32. The system of claim 31 wherein the additional signal produced by one or a combination of labels corresponds to a minor peak in the emission spectrum of a particular chromophore that is not overlapped, or only insignificantly overlapped, by signal from other chromophores.

33. The system of claim 31 wherein the additional signal produced by one or a combination of labels corresponds to a shoulder of a major peak used, in addition a characteristic signal corresponding to the major peak, in order to accurately account for peak broadening or peak shifts due to chromophore-chromophore interactions.

34. The system of claim 31 wherein the additional signal produced by one or a combination of labels corresponds to a secondary emission from a molecule or chemical moiety excited by fluorescent emission from one or more of the number of types of labels.
